(19) United States
(12) Patent Application Publication
(10) Pub. No.: US 2024/0091093 A1
(43) Pub. Date: Mar. 21, 2024

(54) METHOD OF IMPROVING THE CONDITIONS OF AN EYE

(71) Applicant: The Hong Kong University of Science and Technology, Hong Kong (CN)

(72) Inventors: David Chuen Chun LAM, Hong Kong (CN); Yun Yuen LEUNG, Hong Kong (CN)

(21) Appl. No.: 18/469,881

(22) Filed: Sep. 19, 2023

Related U.S. Application Data
(60) Provisional application No. 63/408,113, filed on Sep. 20, 2022.

(51) Int. Cl. A61H 5/00; A61N 5/06

(52) U.S. Cl. CPC A61H 5/00; A61N 5/06; A61H 2201/0207; A61H 2201/10; A61H 2201/1607; A61H 2201/165; A61H 2209/00; A61N 2005/0648; A61N 2005/0659; A61N 2005/0662

(57) ABSTRACT

The subject invention pertains to systems and methods to improve the condition of an eye and to either treat or lower the risk of diseases such as glaucoma. Systems and methods are provided for increasing compliance, improving circulation and fluid flow, or promoting tissue cell metabolism by warm compress, and/or irradiation at single or multiple wavelengths (e.g., between 780 to 950 nm or between 550 to 700 nm), and/or application of static and/or dynamic pressure to the eye. Embodiments provide a wearable head set with pressure controlled goggles, controlled irradiation sources, and temperature-controlled heat sources or pads exposed to or in contact with the eye or surrounding region to warm up the tissues of the eye and promote the vasodilation of the blood vessels at the eye as well as circulation within the blood system connected to the Schlemm's canal or other regions in or around the eye.

### CROSS-REFERENCE TO RELATED APPLICATION

This application claims the benefit of U.S. Provisional Application Ser. No. 63/408,113, filed Sep. 20, 2022, which is hereby incorporated by reference in its entirety including any tables, figures, or drawings.

### BACKGROUND OF THE INVENTION

Glaucoma is an eye disease associated with damage to the optic nerve that can lead to vision loss. The risk of glaucoma increases with age and is related to ethnic or family history. However, there is no authorized preventive measure for patients in high-risk groups. High-risk individuals must rely upon regular eye exams, and treatments can only be started after diagnosis.

Damage to the optic nerve can be caused by excessive intraocular pressure (IOP), resulting in over critical loading stresses in the optic nerve tissues. Lowering the ocular stresses is a crucial approach to slow down glaucoma progression and to inhibit glaucoma from developing in high-risk groups. The ocular stresses are associated with the biomechanical properties of the ocular tissue. The biomechanical property of the ocular tissue is an individual factor and is known to be related to aging. Ocular tissues with higher compliance are desired. High compliance tissues can have higher non-damaging deformation when response to gradual loading from IOP and result in lower accumulation of stresses.

The regulation of the aqueous humor is also essential for IOP control. IOP is primarily controlled by the formation and the drainage of aqueous humor inside an eyeball. The drainage system of an eye is associated with the venous system connected to the Schlemm's canal. Maintaining a healthy condition of the blood circulation and the tissues supporting drainage is essential to inhibit aqueous humor accumulation that can lead to significant elevation of IOP.

High IOP is not an inevitable feature of developing glaucoma. The biomechanical strength of the tissue associated to the optic nerve is also important to resist the damage. The exact mechanisms of deterioration and damage of the ocular tissues are unknown. But the poor vascular circulation of the peripapillary vessel and ineffective tissue cell metabolism are believed to be factors that affect the health of the optic nerve.

Related art technologies have not fully addressed the above concerns. U.S. Pat. No. 6,364,875 to Stanley, III teaches the combined effect of elevated temperature and stretching forces lead to permanent residual strains, reshaping the cornea. U.S. Pat. No. 10,881,550 to Tedford et al. teaches multi-wavelength low level light therapy wherein two or more doses of light are delivered in a coordinated fashion to influence a desired target cell functionality. United States Patent Application Publication 2022/0168136 to BADAWI et al. teaches a patch or strip affixed to the skin of the upper and/or lower eyelids to deliver heat to the one or more meibomian glands contained within the underlying skin for dry eye treatment. Medina (Eye (2017) 31, 1621-1627; doi:10.1038/eye.2017.123) teaches that Pneumatic Keratology can be used to alter the cornea by non-invasive means. A vacuum chamber with radial openings alters the collagen fibers in the stroma and flattens the cornea. Jang et al. (Sci. Adv. 2021; 7: eabf7194 31 Mar. 2021) teaches a soft, smart contact lens and a skin-attachable therapeutic device for wireless monitoring and therapy of chronic ocular surface inflammation (OSI).

### BRIEF SUMMARY OF THE INVENTION

Embodiments of the subject invention provide systems and methods to improve the conditions of an eye and to reduce the risk of suffering from glaucoma. In one embodiment the method comprises warming the tissues of the eye, stretching the tissues of the anterior region of the eye, irradiating the tissue of the drainage system, the optic nerve, and the peripapillary sclera in the eye with infra-red radiation, and irradiating the tissue of the drainage system, the optic nerve, and the peripapillary sclera in the eye with visible light radiation.

Damage to the optic nerve can be caused by excessive intraocular pressure (IOP), resulting in over critical loading stresses in the optic nerve tissues. Lowering the ocular stresses is a crucial approach to slow down glaucoma progression and to inhibit glaucoma from developing in high-risk groups. The ocular stresses are associated with the biomechanical properties of the ocular tissue. The biomechanical property of the ocular tissue is an individual factor and is known to be related to aging. Ocular tissues with higher compliance are desired. High compliance tissues can have higher non-damaging deformation when response to gradual loading from IOP and result in lower accumulation of stresses. Embodiments of the subject invention advantageously provide warming of the ocular tissues to increase compliance and improve condition of the eye.

The regulation of the aqueous humor is also essential for IOP control. IOP is primarily controlled by the formation and the drainage of aqueous humor inside an eyeball. The drainage system of an eye is associated with the venous system connected to the Schlemm's canal. Maintaining a healthy condition of the blood circulation and the tissues supporting drainage is essential to inhibit aqueous humor accumulation that can lead to significant elevation of IOP.

High IOP is not an inevitable feature of developing glaucoma. The biomechanical strength of the tissue associated to the optic nerve is also important to resist the damage. The exact mechanisms of deterioration and damage of the ocular tissues are unknown. But the poor vascular circulation of the peripapillary vessel and ineffective tissue cell metabolism are believed to be factors that affect the health of the optic nerve.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a flowchart showing a method to modify the condition of an eye according to an embodiment of the subject invention.

FIG. 1B is a second flowchart showing method [200], adding irradiation and other steps in a method similar to method according to an embodiment of the subject invention.

FIG. 1C is a third flowchart showing method [300], a modified expansion of method according to an embodiment of the subject invention.

FIGS. 2A-2B are charts showing measurements of intraocular pressure (IOP) in right (FIG. 2A) and left (FIG. 2B) eyes, respectively, over 11 days of treatment according to an embodiment of the subject invention.

FIGS. 3A-3B are charts showing measurements of intraocular pressure (IOP) of one subject in left (FIG. 3A) and right (FIG. 3B) eyes, respectively, over 2 weeks of baseline measurement plus 10 weeks of treatment according to an embodiment of the subject invention under method [300].

FIGS. 3C-3D are charts showing measurements of intraocular pressure (IOP) of another subject in left (FIG. 3C) and right (FIG. 3D) eyes, respectively, over 2 weeks of baseline measurement plus 15 weeks of treatment according to an embodiment of the subject invention under method [300].

FIGS. 4A-4B illustrate the experimental setup according to an embodiment of the subject invention, as used to collect the IOP data in FIGS. 2A-2B and FIGS. 3A-3D.

FIGS. 5A-5B illustrate the first prototype device according to an embodiment of the subject invention.

FIGS. 6A and 6E illustrate the overview of the second prototype device according to an embodiment of the subject invention.

FIGS. 6B and 6F illustrate the heat pad of the second prototype device according to an embodiment of the subject invention.

FIG. 6C illustrates the modular components of the second prototype device according to an embodiment of the subject invention.

FIG. 6D illustrates the second prototype device during operation according to an embodiment of the subject invention.

FIG. 7A illustrates the irradiation spectra centralized at 590 nm wavelength, e.g., for use with Step according to an embodiment of the subject invention.

FIG. 7B illustrates the irradiation spectra centralized at 830 nm wavelength, e.g., for use with Step according to an embodiment of the subject invention.

### DETAILED DISCLOSURE OF THE INVENTION

According to one or more embodiments of the subject invention, there is provided a system and/or method to modify the condition of an eye involving increasing compliance (e.g., in a patient experiencing reduced compliance as a complication of glaucoma) by warm compress of the eye by putting a temperature-controlled heat pad in contact with the eye lid and the periocular region to warm up the tissues of the eye.

As used herein, “subject”, “patient”, “host” or “organism” refers to any member of the phylum Chordata, more preferably any member of the subphylum vertebrata, or most preferably, any member of the class Mammalia, including, without limitation, humans and other primates, including non-human primates such as rhesus macaques, chimpanzees and other monkey and ape species; livestock, such as cattle, sheep, pigs, goats and horses; domestic mammals, such as dogs and cats; laboratory animals, including rabbits, mice, rats and guinea pigs; birds, including domestic, wild, and game birds, such as chickens, turkeys, ducks, and geese. The term does not denote a particular age or gender. Thus, adult, young, and new-born individuals are intended to be covered as well as male and female subjects. In some embodiments, the subject is a non-human subject. In some embodiments, the subject is a human subject. In some embodiments, the subject can be any living creature having eyes suitable for application of the systems and methods disclosed herein.

According to one or more embodiments of the subject invention, there is provided a system and/or method to modify the condition of an eye involving increasing compliance (e.g., in a patient experiencing reduced compliance as a complication of glaucoma) by warm compress of the eye by putting a temperature-controlled heat pad in contact with the periocular region to warm up the tissues of the eye.

According to one or more embodiments of the subject invention, there is provided a system and/or method to modify the condition of an eye and to reduce the risk of suffering from glaucoma, the method involving increasing compliance (e.g., in a patient experiencing reduced compliance as a complication of glaucoma) by stretching the tissue of the cornea and the associated scleral region by applying a static pressure on the periocular region through a goggle device with a pressure-controlled chamber covering the periocular region.

According to one or more embodiments of the subject invention, there is provided a system and/or method to modify the condition of an eye and to reduce the risk of suffering from glaucoma involving increasing compliance (e.g., in a patient experiencing reduced compliance as a complication of glaucoma) by stretching the tissue of the cornea and the associated scleral region by applying a combination of static and dynamic pressure on the periocular region through a goggle device with a pressure-controlled chamber covering the periocular region.

According to one or more embodiments of the subject invention, there is provided a system and/or method to modify the condition of an eye and to reduce the risk of suffering from glaucoma involving promoting circulation within the blood system connected to the Schlemm's canal by warm compress of the eye by putting a temperature-controlled heat pad in contact with the eye lid to promote the vasodilation of the blood vessels at the eye.

According to one or more embodiments of the subject invention, there is provided a system and/or method to modify the condition of an eye and to reduce the risk of suffering from glaucoma involving promoting circulation with the blood system connected to the Schlemm's canal by warm compress of the eye by putting a temperature-controlled heat pad in contact with the periocular region to promote the vasodilation of the blood vessels at the eye.

According to one or more embodiments of the subject invention, there is provided a system and/or method to modify the condition of an eye and to reduce the risk of suffering from glaucoma involving promoting the circulation with the peripapillary vessel by irradiating the anterior and posterior chamber of the eye with infra-red radiation with a single wavelength (e.g., between 780 to 950 nm) or with a combination of peak wavelengths (e.g., between 780 to 950 nm.)

According to one or more embodiments of the subject invention, there is provided a system and/or method to modify the condition of an eye and to reduce the risk of suffering from glaucoma involving promoting circulation with the peripapillary vessel by irradiating the anterior and posterior chamber of the eye with infra-red radiation with a single wavelength (e.g., between 3000 to 12000 nm) or with a combination of wavelengths between 3000 to 12000 nm.

According to one or more embodiments of the subject invention, there is provided a system and/or method to modify the condition of an eye and to reduce the risk of suffering from glaucoma involving promoting tissue cell metabolism by irradiating the anterior and posterior chamber of the eye with visible radiation with single wavelength (e.g., between 550 to 700 nm) or a combination of peak wavelength between 550 to 700 nm.

According to one or more embodiments of the subject invention, any of the aforementioned systems and/or methods can be applied alone or in combination with each other and with other systems and/or methods. Such systems and methods can include therapeutic and diagnostic systems, and treatment for certain conditions or diseases.

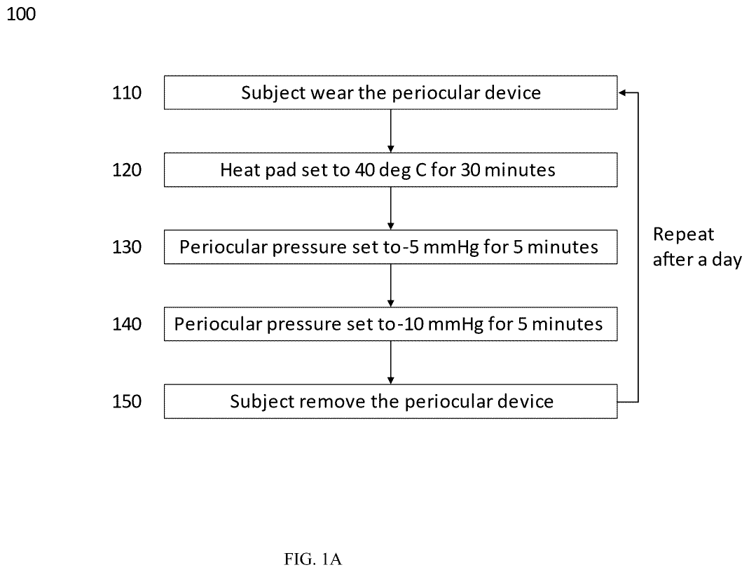

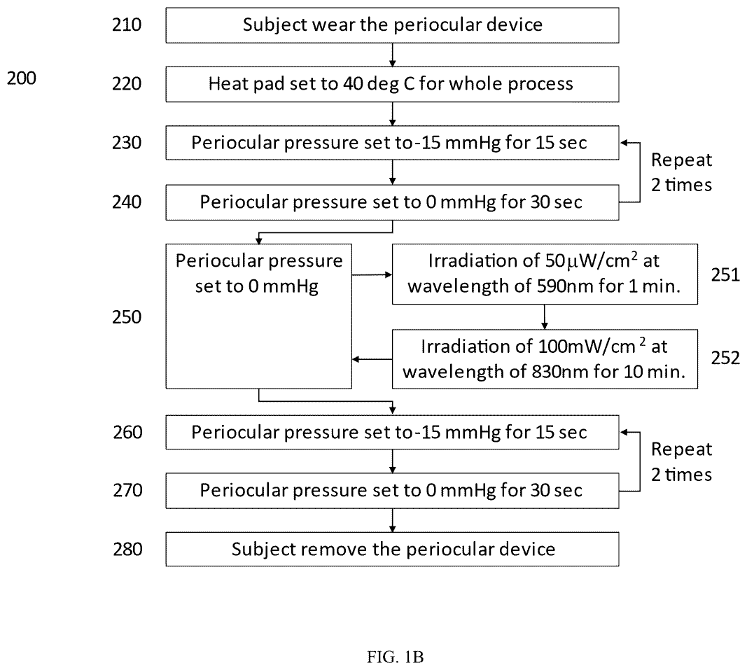

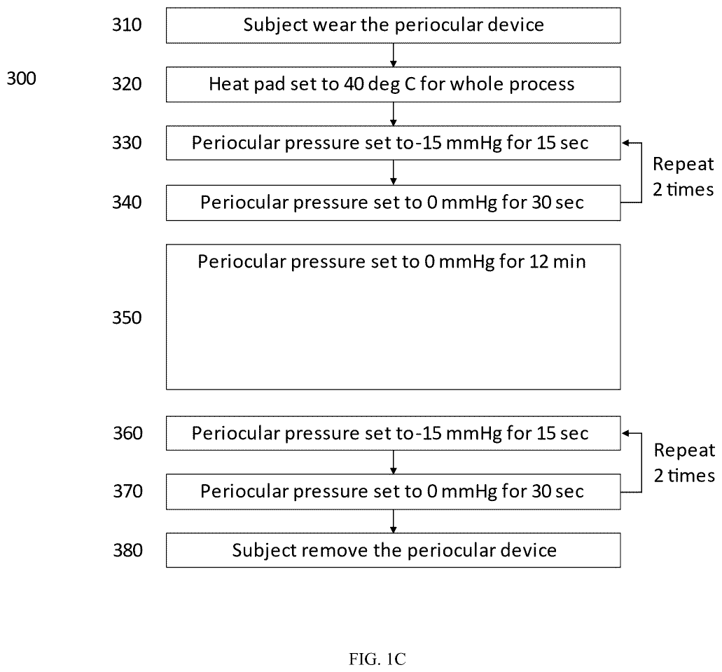

Turning now to the Figures, the embodiment shown in FIG. 1A is an example of a method [100] to modify the eye condition of a patient. The subject can wear, apply, or put on a device in Step [110] that has a temperature-controlled eye pad (e.g., a heat pad) and a pressure chamber covering the periocular region. The heat pad that contacts the eye lid and the periocular skin is set to 40 deg C. for 30 minutes of warm compress in Step [120]. After the warm compress, the pressure level of the pressure chamber that covers the periocular region is set to −5 mmHg for 5 minutes, stretching of the ocular tissue in Step [130]. Then the pressure level of the pressure chamber that covers the periocular region is set to −10 mmHg for 5 minutes of further stretching of the ocular tissues in Step [140]. After the execution, the subject can remove the device in Step and repeat the method after a sufficient rest period (e.g., one day.)

FIG. 1B is a second example of a method for using the present invention method to modify the eye condition of a patient. The subject can wear a device in Step that has a temperature-controlled eye pad (e.g., a heat pad) and a pressure chamber covering the periocular region. The heat pad that contacts the eye lid and the periocular skin is set to 40 deg C. of warm compress for the whole process in Step [220]. During the warm compress, the pressure level of the pressure chamber that covers the periocular region is set to −15 mmHg for 15 seconds, stretching of the ocular tissue in Step [230]. Then the pressure level of the pressure chamber is returned to 0 mmHg for 30 seconds in Step [240]. This pressure cycle in Step to Step between −15 mmHg to 0 mmHg is repeated twice. After the stretching of the ocular tissue, the irradiation of 50 μW/cm2 at the 590 nm wavelength is applied for 1 minute in Step to activate the epidermis and dermis. Then the irradiation of 100 mW/cm2 at the 830 nm wavelength is applied for 10 minutes in Step to penetrate in the eyelids for healing and tissue rejuvenation. After the irradiation, the pressure level of the pressure chamber that covers the periocular region is set to −15 mmHg for 15 seconds, to create stretching of the ocular tissue in Step [260]. Then the pressure level of the pressure chamber is returned to 0 mmHg for 30 seconds in Step [270]. This pressure cycle Step [260] to Step [270] between −15 mmHg to 0 mmHg is repeated twice. After the execution, the subject can remove the device in Step and repeat the method after a sufficient rest period (e.g., one day.)

FIG. 1C is a third example of a method for using the present invention method to modify the eye condition of a patient. The subject can wear a device in Step [310] that has a temperature-controlled eye pad (heat pad) and a pressure chamber covering the periocular region. The heat pad that contacts the eyelid and the periocular skin is set to 40 deg C. of warm compress during the whole process in Step [320]. During the warm compress, the pressure level of the pressure chamber that covers the periocular region is set to −15 mmHg for 15 seconds, stretching of the ocular tissue in Step [330]. Then the pressure level of the pressure chamber is returned to 0 mmHg for 30 seconds in Step [340]. This pressure cycle [Step 330 to 340] between −15 mmHg to 0 mmHg is repeated twice. After the stretching of the ocular tissue, the eyes are warm compress for 12 minutes [Step 350]. After the warm compress, the pressure level of the pressure chamber that covers the periocular region is set to −15 mmHg for 15 seconds, stretching of the ocular tissue in Step [360]. Then the pressure level of the pressure chamber is returned to 0 mmHg for 30 seconds in Step [370]. This pressure cycle [Step 260 to 270] between −15 mmHg to 0 mmHg is repeated twice. After the execution, the subject can remove the device in Step [280] and repeat the method after a sufficient rest period (e.g., one day.)

In embodiments of the subject invention, including but not limited to those illustrated in FIGS. 1A-1C above, additional values and ranges are contemplated. For example, heat can be applied through a heat pad or other means at temperatures from 37.8° C. to 45° C., including increments, combinations, and ranges thereof. Gauge pressures can be applied from −30 mmHg to 0 mmHg, including increments, combinations, and ranges thereof. Irradiation can be applied in visible, infra-red, or other spectra known in the art. Irradiation can be applied at an intensity from 5 μW/cm² to 1 W/cm², including increments, combinations, and ranges thereof. Irradiation can be applied at wavelengths from 550 nm to 12,000 nm, including increments, combinations, and ranges thereof. Pressures, irradiation, and/or temperatures can be applied simultaneously, alternatingly, or in series from 1 to 10 times or more, including increments, combinations, and ranges thereof. Pressures, irradiation and/or temperatures can be applied from 1 second to 1 hour in duration, including increments, combinations, and ranges thereof. Appropriate values, application times, combinations, sequences, and other variables can be selected from the provided ranges to achieve desired effects without undue risk to the patient.

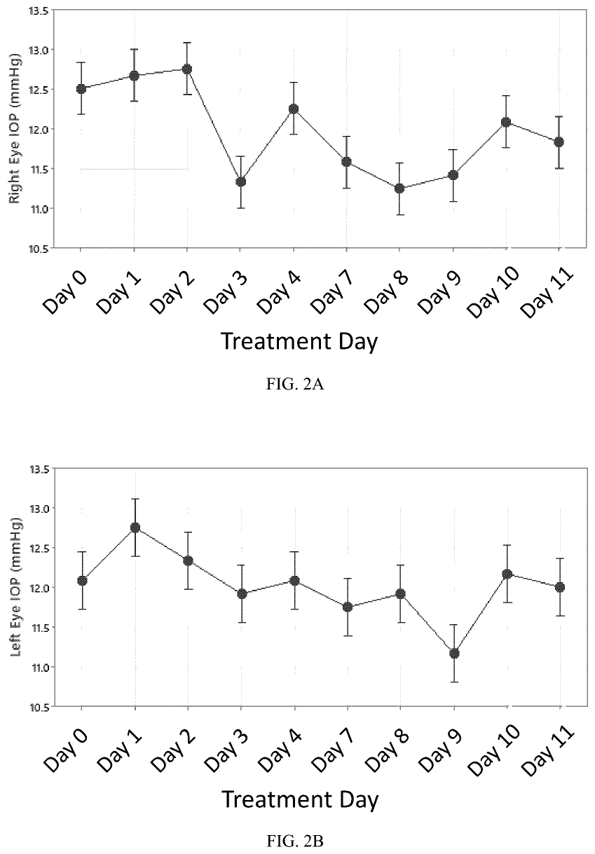

FIGS. 2A-2B show a set of IOP measurement results from a subject after applying treatment according to the embodiment described by method [100]. The IOP of both left eye and right eye was measured using an air-puff tonometer every time before applying the method. There is a decreasing trend of IOP for both eyes after applying the method according to an embodiment of the subject invention.

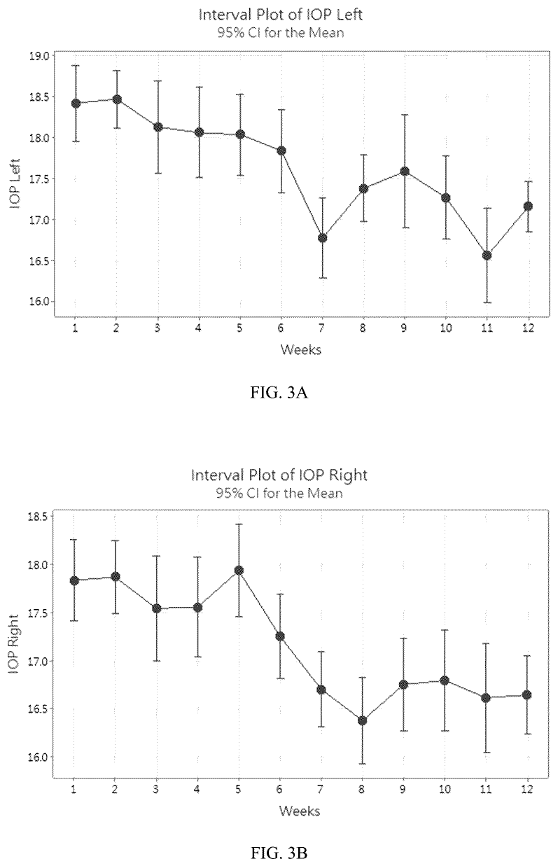

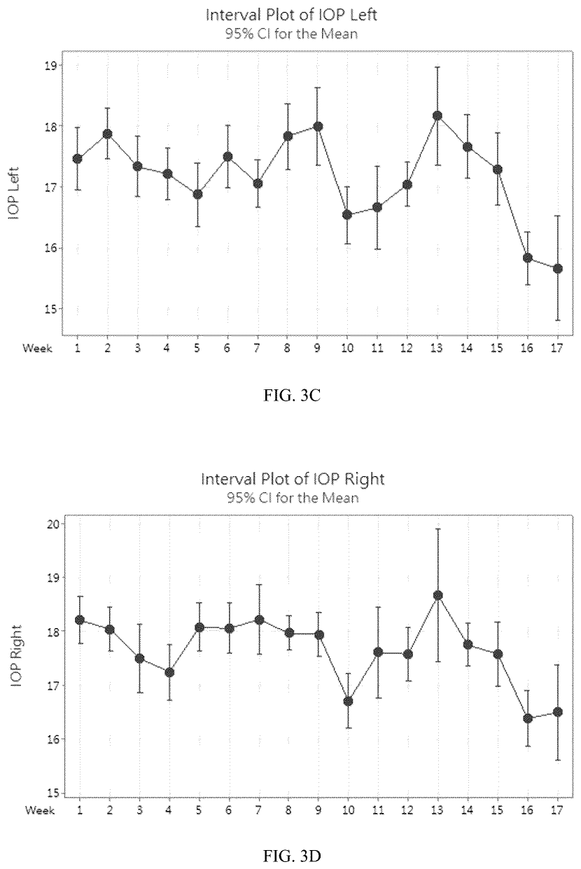

FIGS. 3C-3D are charts showing measurements of intraocular pressure (IOP) of another subject in left (FIG. 3C) and right (FIG. 3D) eyes, respectively, over 15 weeks of treatment according to an embodiment of the subject invention under method [300].

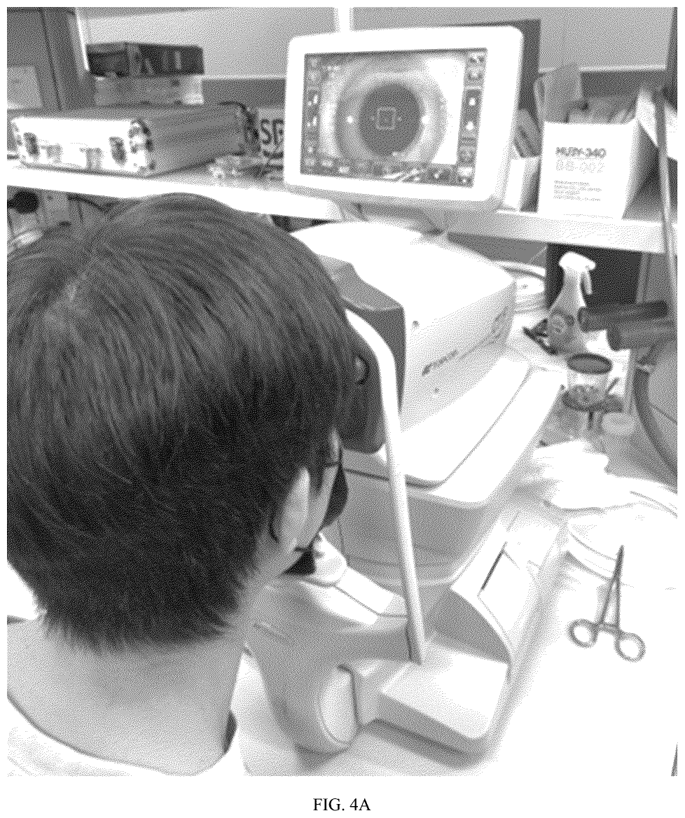

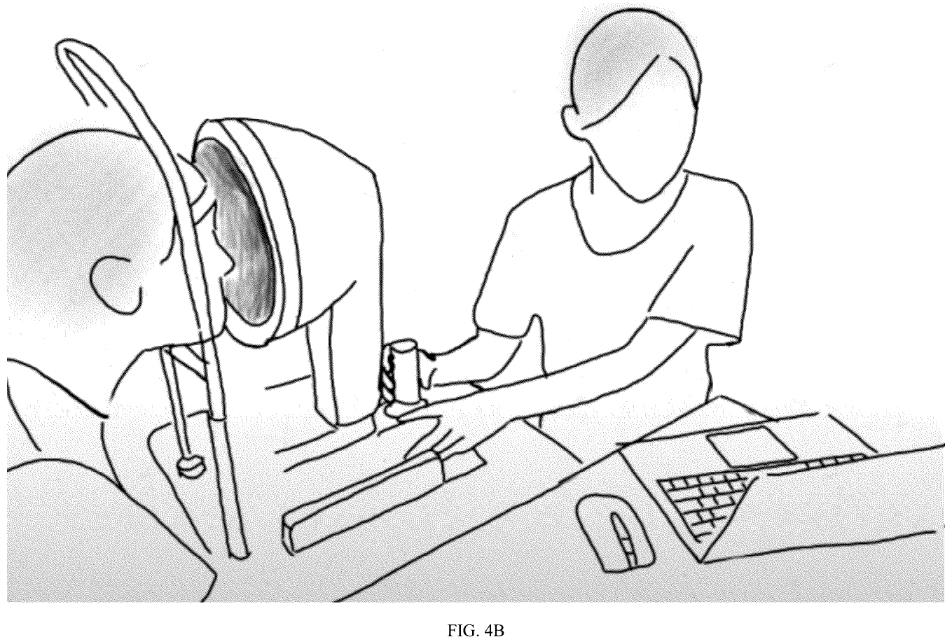

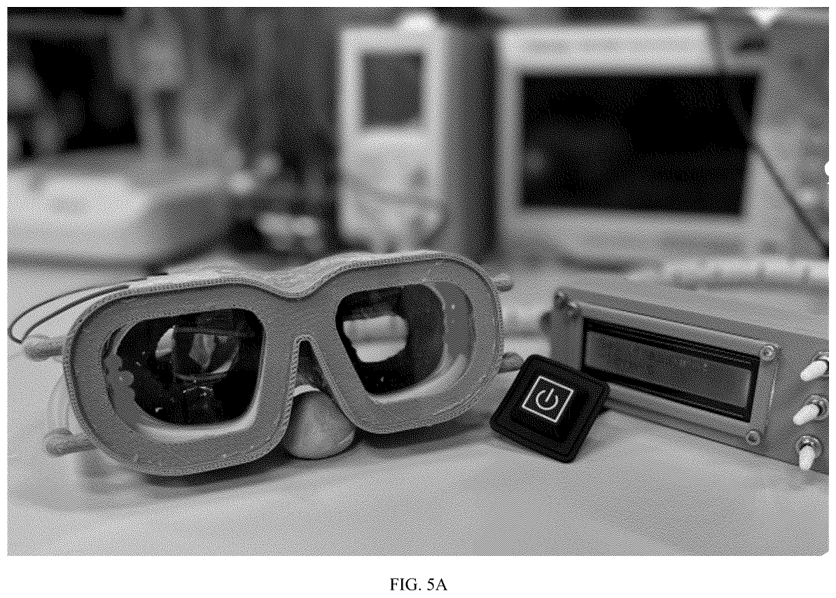

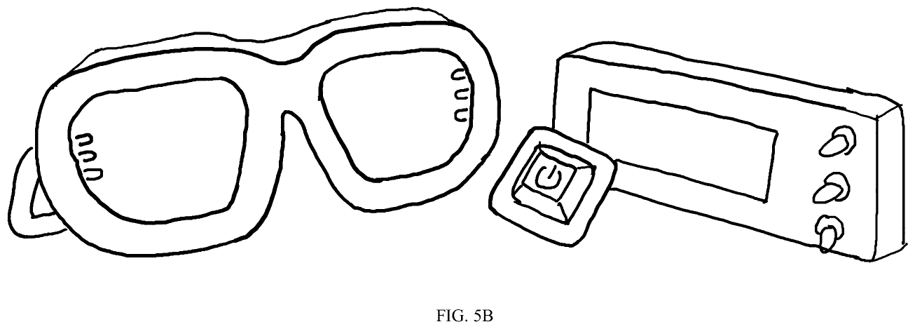

FIGS. 4A-4B illustrate the measurement of intraocular pressure using an air puff tonometer (Topcon CT-1P). The subject was assigned to use the device according to method [100]. The intraocular pressure of the subject was measured the following day after using the device. Before the measurement, the subject was requested to rest for 5 minutes to calm down. The measurement was taken 3 times for each eye, and the data in FIGS. 2A and 2B show the mean and the 95% confident range.

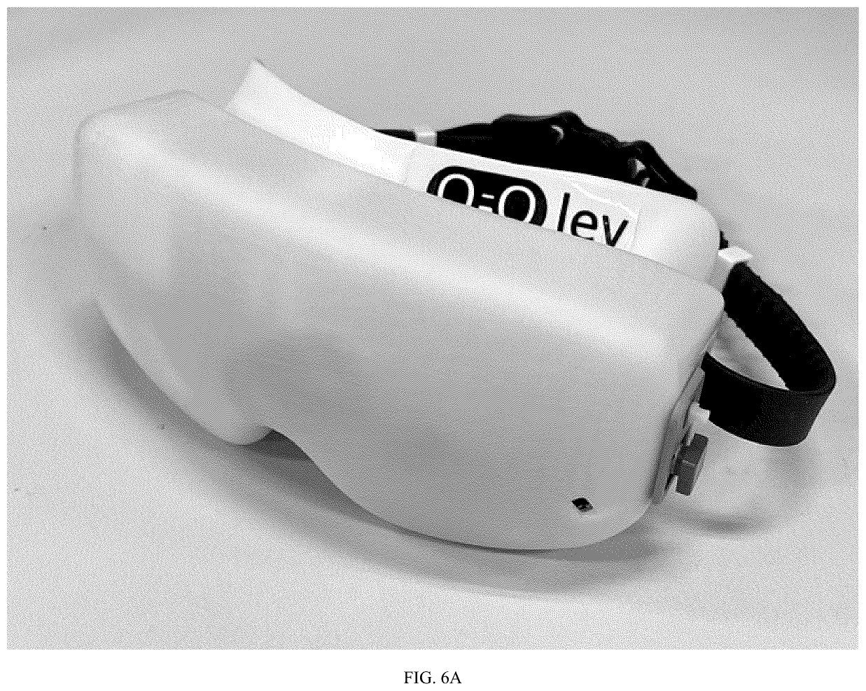

FIGS. 5A-5B illustrate the first prototype of the device. The device consists of a goggle style carrier containing the heat pad module, the pressure control module, and the radiation module. The goggle carrier is connected to a controller through electrical wires and soft piping. The controller contains an electronic circuit, a pumping system, and a power module. The electronics circuit is for monitoring and control of the process. The pumping system is driven to adjust the air flow to control the goggle carrier pressure. The power module is for battery charging and providing power for the device operation.

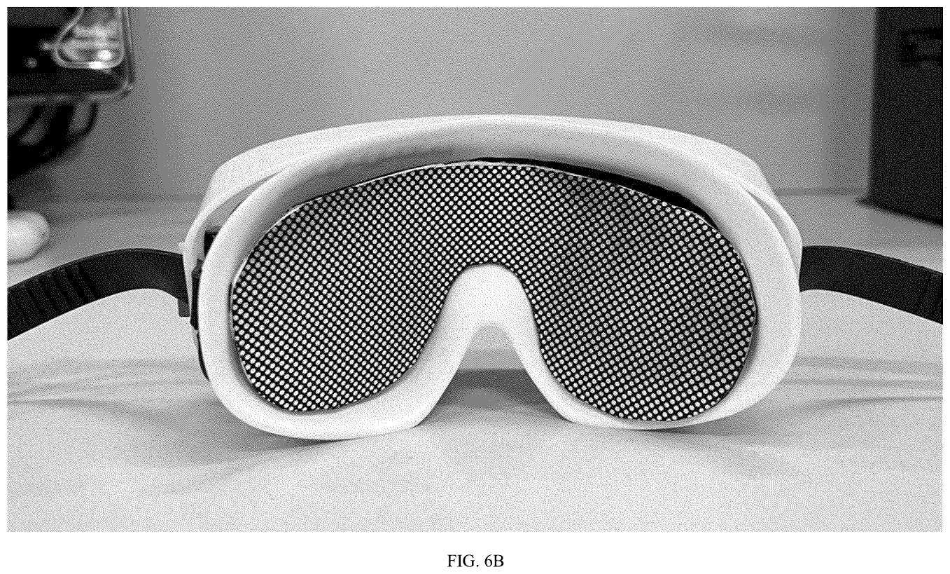

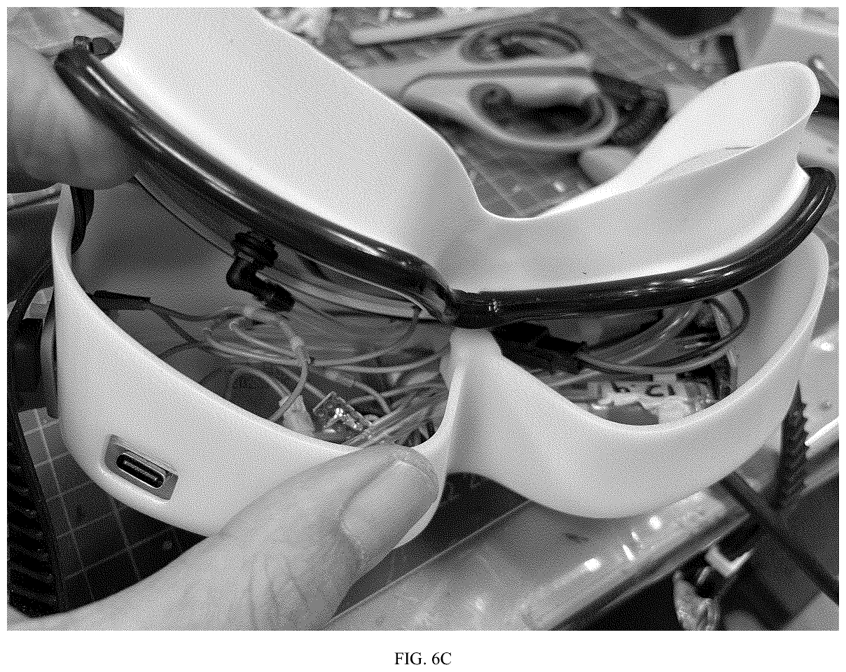

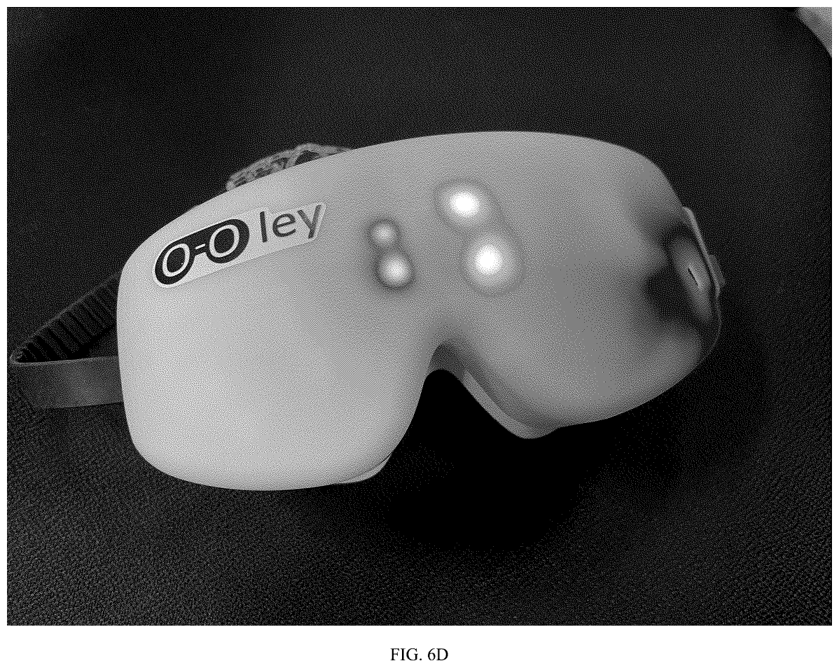

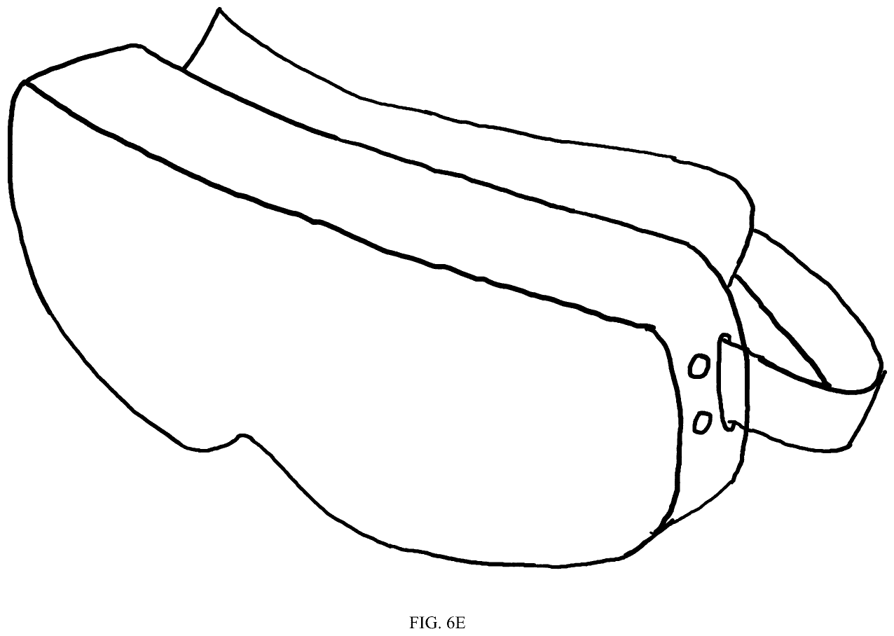

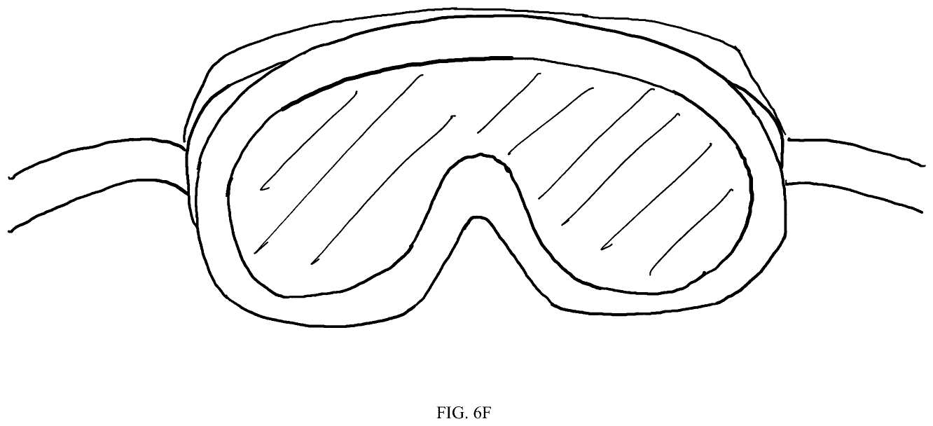

FIGS. 6A and 6E illustrate the second prototype of the device. The device consists of a goggle style carrier containing the heat pad module, the pressure control module, and the radiation module. All the modules are embedded into the carrier.

FIGS. 6B and 6F illustrate the heat pad of the second prototype of the device viewing from the back.

FIG. 6C illustrates the modular components of the second prototype of the device embedded inside the casing.

FIG. 6D illustrates the second prototype of the device during operation with signal indicator light.

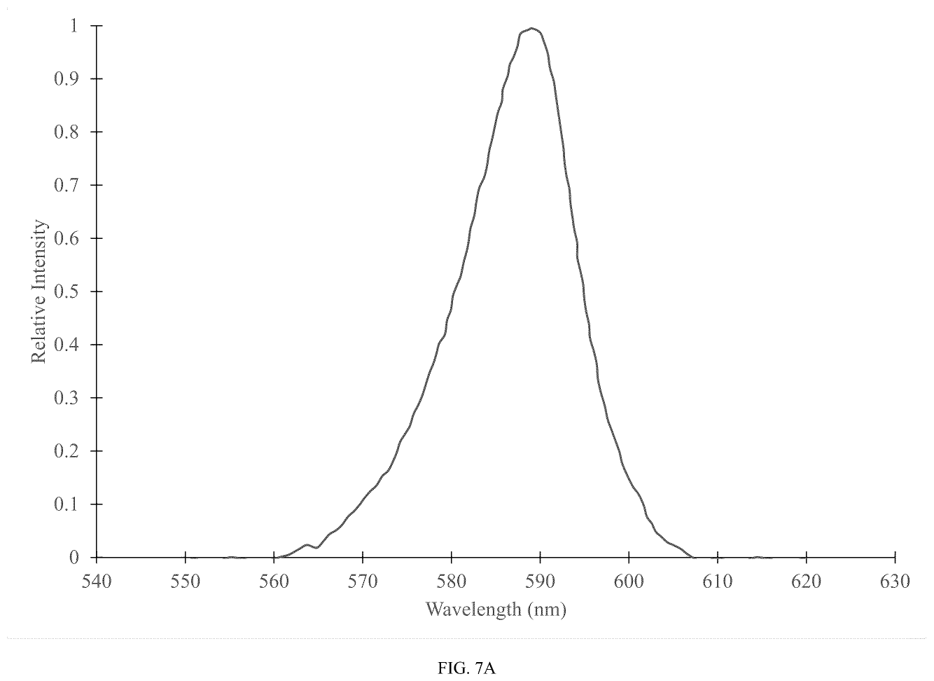

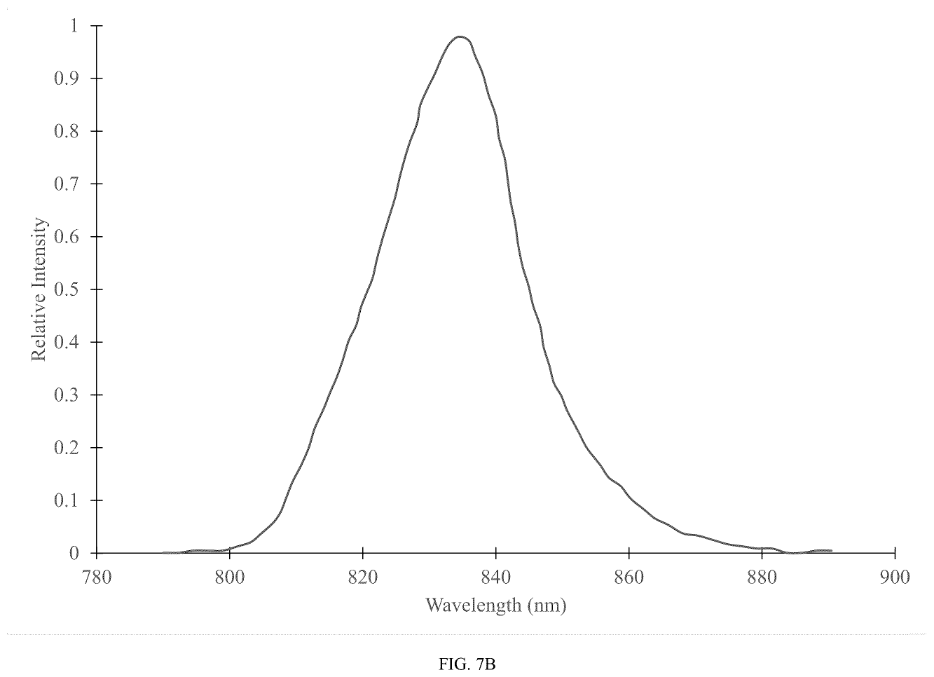

FIG. 7A illustrates the irradiation spectra centralized at 590 nm wavelength for use with [Step 251].

FIG. 7B illustrates the irradiation spectra centralized at 830 nm wavelength for use with [Step 252].

In order that the present disclosure may be more readily understood, certain terms are defined below, and throughout the detailed description, to provide guidance as to their meaning as used herein.

As used herein, the terms “a,” “an,” “the” and similar terms used in the context of the present invention are to be construed to cover both the singular and plural unless otherwise indicated herein or clearly contradicted by the context. Thus, for example, reference to “an arm” or “a hole” should be construed to cover or encompass both a singular arm or a singular hole and a plurality of arms and a plurality of holes, unless indicated otherwise or clearly contradicted by the context.

As used herein, the terms “about” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “and/or” should be understood to mean “either or both” of the features so conjoined, i.e., elements that are conjunctively present in some cases and disjunctively present in other cases.

As used herein, the terms “comprising”, “consisting of” and “consisting essentially of” are defined according to their standard meaning. The terms may be substituted for one another herein in order to attach the specific meaning associated with each term.

As used herein, the term “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The invention may be better understood by reference to certain illustrative examples, including but not limited to the following:
Embodiment 1. A method of improving a condition of an eye in a subject, the method comprising:
warming one or more tissues of the eye; and
stretching one or more tissues of an anterior region of the eye;
irradiating one or more tissues of an optic nerve head and a peripapillary sclera in a posterior chamber of the eye with an infra-red radiation; and
irradiating one or more tissues of the optic nerve head and the peripapillary sclera in the posterior chamber of the eye with a visible light radiation;
thereby improving the condition of the eye in the subject.

Improving the conditions of an eye in a patient can include increasing the vascular circulation. The conditions can include increased blood flow of the blood vessels in the eyeball and the aqueous humor drainage from the anterior chamber of the eye. That can lead to improvements including the strengthening of the ocular tissue and lowering of the intraocular pressure. Improving the conditions of an eye in a patient can also include increasing the compliance of the ocular tissues. The condition includes the increase of the compliance of the cornea tissue and increase of the compliance of the tissue near the optic nerve head. That can lead to the improvement of lowering the intraocular pressure and higher resistance to damage of the optic nerve head under elevated intraocular pressure.

Stretching the tissues of an anterior region of the eye includes applying negative pressure, or a partial vacuum, to the front exterior surface of the eye. This can be accomplished with the eyelid open or closed with pressures ranging from 0 mmHg to −15 mmHg. Other tissues that can be stretched by this or similar methods include the cornea and the eyelid.

Irradiating the tissues of an optic nerve head and a peripapillary sclera in a posterior chamber of the eye with an infra-red radiation means using light source irradiate through the pupil into the posterior chamber of the eye. Useful wavelengths include the range between 780 nm and 950 nm. Times can be ranged from 1 to 15 minutes and intensities can be ranged from 10 to 1000 mW/cm².

Irradiating the tissues of the optic nerve head and the peripapillary sclera in the posterior chamber of the eye with a visible light radiation means using light source irradiate through the pupil into the posterior chamber of the eye. Useful wavelengths include the range between 550 to 700 nm. Times can be ranged from 5 second to 10 minute and intensities can be ranged from 5 to 500 μW/cm².
Embodiment 2. The method according to Embodiment 1, wherein improving the condition of the eye comprises reducing an intraocular pressure (IOP) of the eye.

IOP can be reduced by various methods, including pharmacological agents, laser treatments, and surgery
Embodiment 3. The method according to Embodiment 1, wherein improving the condition of the eye comprises increasing a biomechanical compliance of a cornea of the eye.

Biomechanical compliance is measured by the displacement of the tissue under the applied loading. In addition to the cornea, other tissues comprise the optic nerve head.
Embodiment 4. The method according to Embodiment 1, wherein improving the condition of the eye comprises increasing a vascular circulation connected to a Schlemm's canal of the eye.

Vascular circulation connected to Schlemm's canal means the blood flow connected to the region of Schlemm's canal. Circulation can be increased by increasing the blood flow rate in the blood vessel connected to Schlemm's canal and assisting the aqueous humor carry away by the blood from the Schlemm's canal
Embodiment 5. The method according to Embodiment 1, wherein improving the condition of the eye comprises increasing a vascular circulation of a peripapillary vessel of the eye.

Vascular circulation of a peripapillary vessel means the blood flow of the capillary vessel connected to the peripapillary region. Circulation can be increased by the blood flow rate in the blood vessel connected to the peripapillary region and help provide sufficient nutrients for the surrounding tissues' maintenance.
Embodiment 6. The method according to Embodiment 1, wherein improving the condition of the eye comprises increasing a metabolic rate of one or more groups of cells of the eye.

Increasing a metabolic rate of a group of cells within the eye means increasing the rate of energy expenditure per time for maintaining the normal functions of the cells, which can reduce the deterioration with aging
Embodiment 7. The method according to Embodiment 1, wherein warming the one or more tissues of the eye comprises warming a cornea of the eye.

A cornea can be warmed by the device through the heat transfer by the combination of conduction between the contact of heat pad and eyelids, the convection of the heated air by the heat pad in the chamber of the device, and direct radiation of energy from the heat pad to the eye tissues.
Embodiment 8. The method according to Embodiment 1, wherein warming the one or more tissues of the eye comprises warming the peripapillary sclera of the eye.

The tissues of the peripapillary sclera can be warmed by combining the irradiation of the infra-red radiation through the pupil and the conduction of heat from the anterior chamber to the posterior chamber of the eye.
Embodiment 9. The method according to Embodiment 1, wherein warming the one or more tissues of the eye comprises warming one or more tissues of an extraocular muscle of the eye.

The tissues of an extraocular muscle can be warmed by the skin contact of the heat pad and to the extraocular muscle through conduction.
Embodiment 10. The method according to Embodiment 1, wherein warming the one or more tissues of the eye comprises warming the one or more tissues of the eye to a specified temperature that is higher than a threshold value.

The threshold value can be above 37.8° C. to have beneficial effects. The threshold value can be below 45° C. to avoid skin burn and other potential complications.
Embodiment 11. The method according to Embodiment 10, wherein the threshold value is above a skin surface temperature of the subject.

The skin surface temperature of the patient means the skin surface temperature at the forehead which can be measured by infra-red thermometer, typically in a range from 31.0° C. to 35.6° C.
Embodiment 12. The method according to Embodiment 1, wherein stretching the one or more tissues of the anterior region of the eye comprises stretching a cornea of the eye.
Embodiment 13. The method according to Embodiment 1, wherein stretching the one or more tissues of the anterior region of the eye comprises stretching the peripapillary sclera of the eye.
Embodiment 14. The method according to Embodiment 1, wherein stretching the one or more tissues of the anterior region of the eye comprises changing a periocular pressure of the eye.
Embodiment 15. The method according to Embodiment 1, wherein stretching the one or more tissues of the anterior region of the eye comprises changing an intraocular pressure of the eye.
Embodiment 16. The method according to Embodiment 1, wherein irradiating the one or more tissues of the optic nerve head and the peripapillary sclera in the posterior chamber of the eye with infra-red radiation comprises exposing the one or more tissues to an infra-red radiation source having a peak wavelength between 780 nm and 950 nm.
Embodiment 17. The method according to Embodiment 1, wherein irradiating the one or more tissues of the optic nerve head and the peripapillary sclera in the posterior chamber of the eye with the visible light radiation comprises exposing the one or more tissues to an infra-red radiation source having a wavelength between 3000 nm and 12000 nm.
Embodiment 18. The method according to Embodiment 1, wherein irradiating the one or more tissues of the optic nerve head and the peripapillary sclera in the posterior chamber of the eye with the visible light radiation comprises exposing the tissues to a visible radiation source having a peak wavelength between 550 to 700 nm.
Embodiment 19. A method of improving a condition of an eye in a subject, the method comprising:
warming a cornea, a peripapillary sclera, or an extraocular muscle of the eye to a threshold value that is above a skin surface temperature of the subject; and
stretching the cornea, the peripapillary sclera, or the extraocular muscle of the eye of the eye, thereby changing a periocular pressure of the eye, or an intraocular pressure of the eye, or both;
irradiating one or more tissues of an optic nerve head and the peripapillary sclera in a posterior chamber of the eye with an infra-red radiation having a peak wavelength between 780 nm and 950 nm; and
irradiating one or more tissues of the optic nerve head and the peripapillary sclera in the posterior chamber of the eye with a visible light radiation having a peak wavelength between 550 to 700 nm;
wherein improving the condition of the eye comprises at least one of reducing an intraocular pressure (IOP) of the eye, increasing a biomechanical compliance of the cornea of the eye, increasing a vascular circulation connected to a Schlemm's canal of the eye, increasing a vascular circulation of a peripapillary vessel of the eye, or increasing a metabolic rate of one or more groups of cells of the eye.
Embodiment 20. A device for improving a condition of an eye in a subject, the device comprising:
a goggle carrier;
a heat pad module configured and adapted to warm a cornea, a peripapillary sclera, or an extraocular muscle of the eye to a threshold value that is above a skin surface temperature of the subject;
a pressure control module configured and adapted to stretch the cornea, the peripapillary sclera, or the extraocular muscle of the eye of the eye to an extent sufficient to measurably change a periocular pressure of the eye, or an intraocular pressure of the eye, or both;
a radiation module configured and adapted to irradiate one or more tissues of an optic nerve head and the peripapillary sclera in a posterior chamber of the eye with an infra-red radiation having a peak wavelength between 780 nm and 950 nm and with a visible light radiation having a peak wavelength between 550 to 700 nm; and
a controller operably connected to the heat pad module, the pressure control module, and the radiation module, the controller configured and adapted to provide heat, pressure, and radiation until reaching a desired state comprising a measurable change in at least one of reduction of an intraocular pressure (IOP) of the eye, increase of a biomechanical compliance of the cornea of the eye, an increase of a vascular circulation connected to a Schlemm's canal of the eye, an increase of a vascular circulation of a peripapillary vessel of the eye, and an increase of a metabolic rate of one or more groups of cells of the eye.
Embodiment 21. The device according to Embodiment 20, wherein the goggle carrier is covering a periocular region of the eye.
Embodiment 22. The device according to Embodiment 20, wherein the eye is a first eye and the goggle carrier is covering the periocular region the first eye and also covering a periocular region of a second eye.
Embodiment 23. The device according to Embodiment 20, wherein the heat pad module comprises a heating element layer, a thermal insulation layer, and a soft contact layer.
Embodiment 24. The device according to Embodiment 23, wherein the heating element layer consists of a resistive heating strip embedded in a flexible printed circuit.
Embodiment 25. The device according to Embodiment 24, wherein the resistive heating strip embedded in the flexible printed circuit is powered by a pulse width modulated input voltage regulated by the controller.
Embodiment 26. The device according to Embodiment 25, wherein the thermal insulation layer is in contact with a first side of the heating element layer.
Embodiment 27. The device according to Embodiment 26, wherein the thermal insulation layer comprises a flexible material that conforms to a curved surface on or around the eye.
Embodiment 28. The device according to Embodiment 27, wherein the soft contact layer is in contact with a second side of the heating element layer.
Embodiment 29. The device according to Embodiment 28, wherein the soft contact layer is configured and adapted for contact with a layer of skin on an eyelid covering the eye.
Embodiment 30. The device according to Embodiment 21, wherein the pressure control module comprises a pump.
Embodiment 31. The device according to Embodiment 30, wherein the pressure control module comprises a first cannula fluidly coupling the pump to a cavity of the goggle carrier adjacent the periocular region.
Embodiment 32. The device according to Embodiment 31, wherein the pressure control module comprises a second cannula fluidly coupling the cavity to an area of ambient pressure.
Embodiment 33. The device according to Embodiment 32, wherein the second cannula comprises a check valve configured and adapted to allow fluid flow from the area of ambient pressure into the cavity while inhibiting fluid flow from the cavity into the area of ambient pressure.
Embodiment 34. The device according to Embodiment 33, wherein the check valve is configured and adapted to open when the gauge pressure in the cavity drops below a threshold pressure.
Embodiment 35. The device according to Embodiment 34, wherein the threshold pressure is equal or lower than −10 mmHg.
Embodiment 36. The device according to Embodiment 20, wherein the radiation module comprises a light source configured and adapted to supply the infra-red radiation and the visible light radiation to irradiate an anterior chamber of the eye, the posterior chamber of the eye, and an eyelid of the eye.
Embodiment 37. The device according to Embodiment 36, wherein the light source comprises a visible light emitting element operably connected to a first optical device.
Embodiment 38. The device according to Embodiment 37, wherein the light source comprises an infra-red light emitting element operably connected to a second optical device.
Embodiment 39. The device according to Embodiment 38, wherein the visible light emitting element is powered by a first pulse width modulated input current regulated by the controller.
Embodiment 40. The device according to Embodiment 39, wherein the infra-red light emitting element is powered by a second pulse width modulated input current regulated by the controller.
Embodiment 41. The device according to Embodiment 40, wherein the operable connection between the infra-red light emitting element and the second optical device is configured and adapted to maximize a first radiation luminous intensity distribution output of the infra-red radiation to one or more of the anterior chamber of the eye, the posterior chamber of the eye, and the eyelid of the eye.
Embodiment 42. The device according to Embodiment 41, wherein the controller is configured and adapted to operably couple the regulation of the first pulse width modulated input current and the regulation of the second pulse width modulated input current to deliver a specified total radiation luminous intensity distribution output of the combined visible light radiation and infra-red radiation to one or more of the anterior chamber of the eye, the posterior chamber of the eye, and the eyelid of the eye.
Embodiment 43. The device according to Embodiment 42, wherein the second optical devices comprises or consists of a light guide.
Embodiment 44. The device according to Embodiment 42, wherein the second optical devices comprises or consists of an optical lens.
Embodiment 45. The device according to Embodiment 42, wherein the first optical devices comprises or consists of a light guide.
Embodiment 46. The device according to Embodiment 42, wherein the first optical devices comprises or consists of an optical lens.
Embodiment 47. The method according to Embodiment 1, wherein stretching tissues of the anterior region of the eye comprises stretching the tissue with a blow nose action by the subject.
Embodiment 48. The device according to Embodiment 20, wherein a goggle carrier is covering the periocular region with one eye.
Embodiment 49. The device according to Embodiment 20, wherein a goggle carrier is covering the periocular region with both eyes.
Embodiment 50. The device according to Embodiment 20, wherein a heat pad module consists of a heating element layer, a thermal insulation layer, and a soft contact layer.
Embodiment 51. The device according to Embodiment 50, wherein a heating element layer consists of a resistive heating strip embedded in a flexible printed circuit.
Embodiment 52. The device according to Embodiment 51, wherein a resistive heating strip embedded in a flexible printed circuit is powered by a pulse width modulated input voltage regulated by the controller.
Embodiment 53. The device according to Embodiment 52, wherein a thermal insulation layer on one side is in contact with the heating element layer.
Embodiment 54. The device according to Embodiment 53, wherein a thermal insulation layer consists of flexible material that conform with a curve surface.
Embodiment 55. The device according to Embodiment 54, wherein a soft contact layer on one side is in contact with the heating element layer.
Embodiment 56. The device according to Embodiment 55, wherein a soft contact layer on one side is in contact with the skin on eyelid.
Embodiment 57. The device according to Embodiment 20, wherein a pressure control module consists of a pump.
Embodiment 58. The device according to Embodiment 57, wherein a pressure control module further consists of a first group of one or more cannulas configured for fluidly coupling the pump to a cavity of the goggle carrier at the periocular region.
Embodiment 59. The device according to Embodiment 58, wherein a pressure control module further consists of a second group of one or more cannulas configured for fluidly coupling the cavity of the goggle carrier at the periocular region to the ambient.
Embodiment 60. The device according to Embodiment 59, wherein the second group of one or more cannulas comprises a check valve that only allows fluid flowing from the ambient into the cavity of the goggle carrier at the periocular region.
Embodiment 61. The device according to Embodiment 60, wherein the check valve opens when the gauge pressure in the cavity of the goggle carrier at the periocular region drops below a threshold pressure.
Embodiment 62. The device according to Embodiment 61, wherein a threshold pressure is equal or lower than −10 mmHg.
Embodiment 63. The device according to Embodiment 20, wherein a radiation module consists of a light source configured for directing the radiation on an anterior chamber, a posterior chamber, and an eyelid of an eye.
Embodiment 64. The device according to Embodiment 63, wherein a light source consists of one or plurality of visible light emitting elements and one or plurality of optical devices.
Embodiment 65. The device according to Embodiment 64, wherein a light source consists of one or plurality of infra-red light emitting elements is powered by a pulse width modulated input current regulated by the controller.
Embodiment 66. The device according to Embodiment 65, wherein a light source consists of one or plurality of infra-red light emitting elements and one or plurality of optical devices.
Embodiment 67. The device according to Embodiment 66, wherein a light source consists of one or plurality of infra-red light emitting elements is powered by a pulse width modulated input current regulated by the controller.
Embodiment 68. The device according to Embodiment 67, wherein the one or plurality of visible light emitting elements are coupled with the one or plurality of optical devices have the radiation luminous intensity distribution output approachable on an anterior chamber, a posterior chamber, and an eyelid of an eye.
Embodiment 69. The device according to Embodiment 68, wherein the one or plurality of infra-red light emitting elements are coupled with the one or plurality of optical devices have the radiation luminous intensity distribution output approachable on an anterior chamber, a posterior chamber, and an eyelid of an eye.
Embodiment 70. The device according to Embodiment 69, wherein the optical devices consist of a light guide.
Embodiment 71. The device according to Embodiment 69, wherein the optical devices consist of an optical lens.

Although the present invention has been illustrated and described herein with reference to preferred embodiments and specific examples thereof, it will be readily apparent to those of ordinary skill in the art that other embodiments and examples may perform similar functions and/or achieve like results.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Materials and Methods

All patents, patent applications, provisional applications, and publications referred to or cited herein are incorporated by reference in their entirety, including all figures and tables, to the extent they are not inconsistent with the explicit teachings of this specification.

The following are examples that illustrate procedures for practicing the invention. These examples should not be construed as limiting. All percentages are by weight and all solvent mixture proportions are by volume unless otherwise noted.

### Example 1

An example of a method for using the present invention method to modify the eye condition of a patient is shown in FIGS. 1A-1C. The subject can wear a device in Step that has a temperature-controlled eye pad (e.g., a heat pad) and a pressure chamber covering the periocular region. The heat pad that contacts the eye lid and the periocular skin is set to 40 deg C. for 30 minutes of warm compress in Step [120]. After the warm compress, the pressure level of the pressure chamber that covers the periocular region is set to −5 mmHg for 5 minutes, stretching of the ocular tissue in Step [130]. Then the pressure level of the pressure chamber that covers the periocular region is set to −10 mmHg for 5 minutes of further stretching of the ocular tissues in Step [140]. After the execution, the subject can remove the device in Step and repeat the method after a sufficient rest period (e.g., one day.) The experiment is conducted daily at the same hour for a week to investigate the trend of the IOP. Before the IOP measurement, the subject was asked to rest for 5 minutes to calm down. The test apparatus for measurements of IOP is shown in FIGS. 4A-4B. The subjects must place their head on the tonometer holder and their forehead touches the front holder. The tonometer shot an air puff to measure the IOP of the right eye of the subject three times. The measurement was repeated for measurement of the left eye. To verify the effect of the device, a trend of weekly IOP of the right eye and the left eye were shown in FIG. 2A and FIG. 2B respectively to observe the daily differences. The daily IOPs for both eyes show a decreasing trend. The IOP of the left eye was decreased by 6.96% and the right eye was decreased by 3.55%. after using the device for 7 days.

### Example 2

Another example of a method for using the present invention method to modify the eye condition of a patient. The subject can wear a device in Step that has a temperature-controlled eye pad (e.g., a heat pad) and a pressure chamber covering the periocular region. The heat pad that contacts the eyelid and the periocular skin is set to 40 deg C. of warm compress during the entire process in Step [320]. During the warm compress, the pressure level of the pressure chamber that covers the periocular region is set to −15 mmHg for 15 seconds, stretching of the ocular tissue in Step [330]. Then the pressure level of the pressure chamber is returned to 0 mmHg for 30 seconds in Step [340]. This pressure cycle Step to Step between −15 mmHg to 0 mmHg is repeated twice. After the stretching of the ocular tissue, the eyes are warm compressed for 12 minutes Step [350]. After the warm compress, the pressure level of the pressure chamber that covers the periocular region is set to −15 mmHg for 15 seconds, stretching of the ocular tissue in Step [360]. Then the pressure level of the pressure chamber is returned to 0 mmHg for 30 seconds in Step [370]. This pressure cycle Step to Step between −15 mmHg to 0 mmHg is repeated twice. After the execution, the subject can remove the device in Step and repeat the method after a sufficient rest period (e.g., one day.) The experiment is conducted daily at the same hour for a week to investigate the trend of the IOP. Before the IOP measurement, the subject was asked to rest for 5 minutes to calm down.

The experimental setup for measurements of IOP is shown in FIGS. 4A-4B. The subjects must place their head on the tonometer holder and their forehead touches the front holder. The tonometer shot an air puff to measure the IOP of the right eye of the subject three times. The measurement was repeated for measurement of the left eye. To verify the effect of the device, a trend of weekly IOP of the right eye and the left eye were shown in FIG. 3A and FIG. 3B respectively to observe the weekly differences for 12 weeks. The IOP data for the first two weeks were the baseline IOP and no device usage during the period and one month prior. The IOP data, starting from week 3 to week 12, are the IOP measurement with the daily usage of the device following method [300].

For one subject, there were 54 measurements for the baseline IOP and 242 for the TOP during the device's usage. The baseline mean average for the left eye and right eye IOP were 18.44 mmHg and 17.85 mmHg respectively. The mean average for left eye and right eye IOP at the end of using the device were 17.45 mmHg and 17.02 mmHg respectively. The decrease in mean TOP for left eye and right eye are 0.99 mmHg and 0.83 mmHg respectively. The respective P-values for comparing the IOP baseline and IOP at the end of device usage for both left eye and right eye were calculated as effectively 0, meaning that the IOP for each eye before and after using the device for 10 weeks were significantly different.

For another subject, there were 66 measurements for the baseline IOP and 276 for the TOP during the device's usage. The baseline mean average for the left eye and right eye IOP were 17.55 mmHg and 17.89 mmHg respectively. The mean average for left eye and right eye IOP at the end of using the device were 17.17 mmHg and 17.64 mmHg respectively. The decrease in mean TOP for left eye and right eye are 0.38 mmHg and 0.25 mmHg respectively. The P-value for comparing the baseline and IOP at the end of device usage for left eye and right eye were 0.031 and 0.136 respectively. Therefore, the IOP of the left eye before and after using the device for 15 weeks were significantly different.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method of improving a condition of an eye in a subject, the method comprising:
warming one or more tissues of the eye; and
stretching one or more tissues of an anterior region of the eye;
irradiating one or more tissues of an optic nerve head and a peripapillary sclera in a posterior chamber of the eye with an infra-red radiation; and
irradiating one or more tissues of the optic nerve head and the peripapillary sclera in the posterior chamber of the eye with a visible light radiation;
thereby improving the condition of the eye in the subject.

2. The method according to claim 1, wherein improving the condition of the eye comprises reducing an intraocular pressure (IOP) of the eye.

3. The method according to claim 1, wherein improving the condition of the eye comprises increasing a biomechanical compliance of a cornea of the eye.

4. The method according to claim 1, wherein improving the condition of the eye comprises increasing a vascular circulation connected to a Schlemm's canal of the eye.

5. The method according to claim 1, wherein improving the condition of the eye comprises increasing a vascular circulation of a peripapillary vessel of the eye.

6. The method according to claim 1, wherein improving the condition of the eye comprises increasing a metabolic rate of one or more groups of cells of the eye.

7. The method according to claim 1, wherein warming the one or more tissues of the eye comprises warming a cornea of the eye.

8. The method according to claim 1, wherein warming the one or more tissues of the eye comprises warming the peripapillary sclera of the eye.

9. The method according to claim 1, wherein warming the one or more tissues of the eye comprises warming one or more tissues of an extraocular muscle of the eye.

10. The method according to claim 1, wherein warming the one or more tissues of the eye comprises warming the one or more tissues of the eye to a specified temperature that is higher than a threshold value.

11. The method according to claim 10, wherein the threshold value is above a skin surface temperature of the subject.

12. The method according to claim 1, wherein stretching the one or more tissues of the anterior region of the eye comprises stretching a cornea of the eye.

13. The method according to claim 1, wherein stretching the one or more tissues of the anterior region of the eye comprises stretching the peripapillary sclera of the eye.

14. The method according to claim 1, wherein stretching the one or more tissues of the anterior region of the eye comprises changing a periocular pressure of the eye.

15. The method according to claim 1, wherein stretching the one or more tissues of the anterior region of the eye comprises changing an intraocular pressure of the eye.

16. The method according to claim 1, wherein irradiating the one or more tissues of the optic nerve head and the peripapillary sclera in the posterior chamber of the eye with infra-red radiation comprises exposing the one or more tissues to an infra-red radiation source having a peak wavelength between 780 nm and 950 nm.

17. The method according to claim 1, wherein irradiating the one or more tissues of the optic nerve head and the peripapillary sclera in the posterior chamber of the eye with the visible light radiation comprises exposing the one or more tissues to an infra-red radiation source having a wavelength between 3000 nm and 12000 nm.

18. The method according to claim 1, wherein irradiating the one or more tissues of the optic nerve head and the peripapillary sclera in the posterior chamber of the eye with the visible light radiation comprises exposing the tissues to a visible radiation source having a peak wavelength between 550 to 700 nm.

19. A method of improving a condition of an eye in a subject, the method comprising:
warming a cornea, a peripapillary sclera, or an extraocular muscle of the eye to a threshold value that is above a skin surface temperature of the subject; and
stretching the cornea, the peripapillary sclera, or the extraocular muscle of the eye of the eye, thereby changing a periocular pressure of the eye, or an intraocular pressure of the eye, or both;
irradiating one or more tissues of an optic nerve head and the peripapillary sclera in a posterior chamber of the eye with an infra-red radiation having a peak wavelength between 780 nm and 950 nm; and
irradiating one or more tissues of the optic nerve head and the peripapillary sclera in the posterior chamber of the eye with a visible light radiation having a peak wavelength between 550 to 700 nm;
wherein improving the condition of the eye comprises at least one of reducing an intraocular pressure (IOP) of the eye, increasing a biomechanical compliance of the cornea of the eye, increasing a vascular circulation connected to a Schlemm's canal of the eye, increasing a vascular circulation of a peripapillary vessel of the eye, or increasing a metabolic rate of one or more groups of cells of the eye.

20. A device for improving a condition of an eye in a subject, the device comprising:
a goggle carrier;
a heat pad module configured and adapted to warm a cornea, a peripapillary sclera, or an extraocular muscle of the eye to a threshold value that is above a skin surface temperature of the subject;
a pressure control module configured and adapted to stretch the cornea, the peripapillary sclera, or the extraocular muscle of the eye of the eye to an extent sufficient to measurably change a periocular pressure of the eye, or an intraocular pressure of the eye, or both;
a radiation module configured and adapted to irradiate one or more tissues of an optic nerve head and the peripapillary sclera in a posterior chamber of the eye with an infra-red radiation having a peak wavelength between 780 nm and 950 nm and with a visible light radiation having a peak wavelength between 550 to 700 nm; and
a controller operably connected to the heat pad module, the pressure control module, and the radiation module, the controller configured and adapted to provide heat, pressure, and radiation until reaching a desired state comprising a measurable change in at least one of reduction of an intraocular pressure (IOP) of the eye, increase of a biomechanical compliance of the cornea of the eye, an increase of a vascular circulation connected to a Schlemm's canal of the eye, an increase of a vascular circulation of a peripapillary vessel of the eye, and an increase of a metabolic rate of one or more groups of cells of the eye.
